(19) United States
(12) Patent Application Publication
(10) Pub. No.: US 2010/0172801 A1
(43) Pub. Date: Jul. 8, 2010

(54) METHOD FOR UNIFORM APPLICATION OF FLUID INTO A REACTIVE REAGENT AREA

(76) Inventors: Michael J. Pugia, Granger, IN (US); James A. Profitt, Goshen, IN (US); Hai-Hang Kuo, Granger, IN (US); Gert Blankenstein, Dortmund (DE); Ralf-Peter Peters, Bergisch Gladbach (DE); Lloyd S. Schulman, Osceola, IN (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/688,346

(22) Filed: Jan. 15, 2010

Related U.S. Application Data
(63) Continuation of application No. 10/608,400, filed on Jun. 27, 2003, now abandoned.

(51) Int. Cl. G01N 30/00; G01N 33/487

(52) U.S. Cl. 422/69; 422/68.1

(57) ABSTRACT

Analytical results obtained with microfluidic devices are improved by providing structural features in areas containing dry supported reagents, the structural features directing the flow of a sample over the area in a predetermined uniform manner and facilitating the purging of air.

This application claims benefit of patent application U.S. Ser. No. 10/608,400 filed Jun. 27, 2003.

### BACKGROUND OF THE INVENTION

This invention relates to microfluidic devices, particularly those that are used for analysis of biological samples. Such devices are intended to accept very small samples of blood, urine, and the like. The samples are brought into contact with reagents capable of indicating the presence and quantity of analytes found in the sample. Microfluidic devices are intended to be used for rapid analysis, thus avoiding the delay inherent in sending a biological sample to a central laboratory.

Many devices have been suggested for analysis near the patient, some of which will be discussed below. In general, such devices use only small samples, typically 0.1 to 200 μL. With the development of microfluidic devices the samples required have become smaller typically about 0.1 to 20 μL, which is a desirable aspect of their use. However, smaller samples introduce difficult problems. If accurate and repeatable results are to be obtained, the amount of the sample must be accurately measured and delivered to the reagent. Particularly, when the reagent is dry, e.g. deposited on a substrate, distributing the sample to the supported reagent and purging air from the reaction chamber are critical factors. The present invention addresses these and other problems and provides a means for uniformly contacting a sample fluid with a reagent.

Many prior devices used capillary passageways to transfer a sample to a reagent area, the excess sample being drawn off into separate spaces. Typically, these devices contained reagent chambers which defined the amount of the reagent present. It was presumed that the amount of the sample which contacted the reagent was correct and that the distribution of the sample was uniform. Whether or not such devices provided accurate and repeatable results, it has been found that as the size of the sample to be analyzed becomes very small, say below about 2 μL, obtaining the desired performance becomes more and more difficult.

Blatt et al, U.S. Pat. No. 4,761,381 describes a device used for samples of about 5-10 μL. A portion of the sample fills the reagent chamber, while excess is drawn off through a capillary passageway into a adjacent space. No means for distributing the sample is provided, which is presumed to fill the reagent chamber when air has been purged through a vent.

Charlton et al, U.S. Pat. No. 5,208,163, describes a similar device for use with samples of about 2 μL or more. Again, a portion of a sample is delivered to a reagent area, with the excess being drawn off through a capillary. One feature of the device is the use of a fiber pad to filter out the red blood cells from samples of whole blood. However, there is no attempt made to uniformly distribute the sample over the reagent region.

Weigl, U.S. 2001/0046453, a published patent application, describes a device used for blood typing. Small samples are contacted with liquid reagents and reaction occurs while they are passing through a capillary passage into a waste chamber. Such a device has no reaction chamber of the sort provided in the patents discussed above.

Kellogg et al, U.S. Pat. No. 6,063,589, contains an extended discussion of microfluidic devices for analysis of small samples but does not address the problems relating to assuring that a sample fluid is uniformly distributed over a reagent area.

Musho et al, U.S. Pat. Nos. 5,202,261 and 5,250,439, say that their device is useful for samples of less than 1 μL. The sample being analyzed is passed through a capillary over a region containing the reagent, but does not meter the amount of sample. No means is provided to assure that the sample is uniformly distributed over the reagent area.

Nilsson et al., U.S. Pat. No. 5,286,454, describes a cuvette for analyzing a sample by mixing it with a liquid reagent. Contacting a small liquid sample with a dry reagent is not discussed.

Shanks et al., U.S. Pat. No. 5,141,868, discloses an electrochemical device in which a sample is drawn into capillary passages for measurement. Contact of the sample with dry reagents is not involved in the device.

Moore, U.S. Pat. No. 5,141,868, describes a device in which a sample is subdivided and distributed onto reagent pads by multiple capillaries. Although dry reagents are used, there is no distribution over the pads except that provided by the capillaries.

Blatt et al., EP 287,883, discloses a device similar in concept to Blatt et al's '381 U.S. patent in that a sample is provided to a reagent area, while a capillary passage removes the excess sample. As before, no provision is made for uniform distribution of the sample over a dry reagent.

Tan et al in Anal. Chem. 1999, 71, 1464-1468, describes microfabricated filters for use where particles must be removed from a small sample, e.g. red blood cells from whole blood. The microfilter structures were to be included in a microfluidic device. The article was not concerned with contacting of samples after filtration with dry reagents.

One of the inventions disclosed in U.S. Pat. No. 6,296,126 is the use of wedge-shaped cutouts to assist removing liquid from a capillary and collected in a collection chamber as a free-flowing liquid.

The present inventors have found that, when very small samples are used in a microfluidic device, it is important to provide means for contacting the sample with dry reagents. Their method of doing so is described in detail below.

### SUMMARY OF THE INVENTION

The invention relates in particular to the use in a microfluidic device of microstructures adapted to uniformly distribute small samples of 10 μL or less over reagents disposed on a substrate, thereby making possible accurate and repeatable assays of the analytes of interest in such samples.

In one aspect, the invention is a microfluidic device including such microstructures to facilitate contacting of small samples with a reagent. Referring to FIG. 5A, one preferred microstructure is an array of posts 35a aligned to distribute the sample over the substrate 40 containing the reagent. The array of posts 35a may be in a series of staggered columns aligned at a right angle to the general direction of sample flow. In some embodiments, the posts may be configured to direct flow toward the reagent. For example, the posts may contain wedge-shaped cutouts aligned vertically to the substrate containing the reagent. Referring now to FIG. 5B, other useful microstructures include grooves or weirs 35b disposed at a right angle to sample flow to distribute liquid flow in a uniform front. Ramps may be provided over which samples flow upward to reagents placed on a plateau.

One embodiment of the invention is a microfluidic device for assaying the amount of glycated hemoglobin in a sample of blood. Another embodiment is a microfluidic device for assaying the amount of glucose in a blood sample.

In another aspect, the invention is a method for distributing a small liquid sample of 10 μL or less over a reagent disposed on a substrate.

In some embodiments, the invention is a method of introducing a liquid sample to an elongated absorbent strip for carrying out a sequence of reactions.

### BRIEF DESCRIPTION OF THE DRAWINGS

FIG. 1 illustrates a microfluidic chip of Example 1.

FIG. 2 illustrates a microfluidic chip of Example 2.

FIG. 3 shows a cross-sectional view of the microfluidic chip of Example 4.

FIG. 4 illustrates microstructures used in the microfluidic chip of Example 4.

FIGS. 5A and 5B illustrate microstructures used with a substrate in a more basic microfluidic chip design.

### DESCRIPTION OF THE PREFERRED EMBODIMENTS

### Flow in Microchannels

The devices employing the invention typically use smaller channels than have been proposed by previous workers in the field. In particular, the channels used in the invention have widths in the range of about 10 to 50 μm, preferably about 20-100 μm, whereas channels an order of magnitude larger have typically been used by others when capillary forces are used to move fluids. The minimum dimension for such channels is believed to be about 5 μm since smaller channels may effectively filter out components in the sample being analyzed. Channels of the size preferred in the invention make it possible to move liquid samples by capillary forces alone. It is also possible to stop movement by capillary walls that have been treated to become hydrophobic relative to the sample fluid. The resistance to flow can be overcome by applying a pressure difference, for example, by pumping, vacuum, electroosmosis, heating, absorbent materials, additional capillarity or centrifugal force. As a result, liquids can be metered and moved from one region of the device to another as required for the analysis being carried out.

A mathematical model can be used to relate the centrifugal force, the fluid physical properties, the fluid surface tension, the surface energy of the capillary walls, the capillary size and the surface energy of particles contained in fluids to be analyzed. It is possible to predict the flow rate of a fluid through the capillary and the desired degree of hydrophobicity or hydrophilicity. The following general principles can be drawn from the relationship of these factors.

For any given passageway, the interaction of a liquid with the surface of the passageway may or may not have a significant effect on the movement of the liquid. When the surface to volume ratio of the passageway is large i.e. the cross-sectional area is small, the interactions between the liquid and the walls of the passageway become very significant. This is especially the case when one is concerned with passageways with nominal diameters less than about 200 μm, when capillary forces related to the surface energies of the liquid sample and the walls predominate. When the walls are wetted by the liquid, the liquid moves through the passageway without external forces being applied. Conversely, when the walls are not wetted by the liquid, the liquid attempts to withdraw from the passageway. These general tendencies can be employed to cause a liquid to move through a passageway or to stop moving at the junction with another passageway having a different cross-sectional area. If the liquid is at rest, then it can be moved by a pressure difference, such as by applying centrifugal force. Other means could be used, including air pressure, vacuum, electroosmosis, heating, absorbent materials, additional capillarity and the like, which are able to induce the needed pressure change at the junction between passageways having different cross-sectional areas or surface energies. In the present invention the passageways through which liquids move are smaller than have been used heretofore. This results in higher capillary forces being available and makes it possible to move liquids by capillary forces alone, without requiring external forces, except for short periods when a capillary stop must be overcome. However, the smaller passageways inherently are more likely to be sensitive to obstruction from particles in the biological samples or the reagents. Consequently, the surface energy of the passageway walls is adjusted as required for use with the sample fluid to be tested, e.g. blood, urine, and the like. This feature allows more flexible designs of analytical devices to be made. The devices can be smaller than the disks that have been used in the art and can operate with smaller samples. However, using smaller samples introduces new problems that are overcome by the present invention. For example, air trapped in the device can lead to underfilling or can interfere with liquid handling steps downstream. Of particular importance is the distribution of liquid samples onto substrates containing reagents.

### Microfluidic Analytical Devices

The analytical devices of the invention may be referred to as “chips”. They are generally small and flat, typically about 1 to 2 inches square (25 to 50 mm square) or disks having a radius of about 40 to 80 mm. The volume of samples will be small. For example, they will contain only about 0.1 to 10 μL for each assay, although the total volume of a specimen may range from 10 to 200 μL. The wells for the sample fluids will be relatively wide and shallow in order that the samples can be easily seen and changes resulting from reaction of the samples can be measured by suitable equipment. The interconnecting capillary passageways typically will have a width in the range of 10 to 500 μm, preferably 20 to 100 μm, and the shape will be determined by the method used to form the passageways. The depth of the passageways should be at least 5 μm.

While there are several ways in which the capillaries and sample wells can be formed, such as injection molding, laser ablation, diamond milling or embossing, it is preferred to use injection molding in order to reduce the cost of the chips. Generally, a base portion of the chip will be cut to create the desired network of sample wells and capillaries and then, after reagents have been placed in the wells as desired, a top portion will be attached over the base to complete the chip.

The chips are intended to be disposable after a single use. Consequently, they will be made of inexpensive materials to the extent possible, while being compatible with the reagents and the samples which are to be analyzed. In most instances, the chips will be made of plastics such as polycarbonate, polystyrene, polyacrylates, or polyurethane; alternatively, they can be made from silicates, glass, wax or metal.

The capillary passageways will be adjusted to be either hydrophobic or hydrophilic, properties which are defined with respect to the contact angle formed at a solid surface by a liquid sample or reagent. Typically, a surface is considered hydrophilic if the contact angle is less than 90 degrees and hydrophobic if the contact angle is greater than 90°. It is preferred that the surface energy of the capillary walls is adjusted, i.e. the degree of hydrophilicity or hydrophobicity, for use with the intended sample fluid. For example, to prevent deposits on the walls of a hydrophobic passageway or to assure that none of the liquid is left in a passageway. Preferably, plasma induced polymerization is carried out at the surface of the passageways to adjust the contact angle. Other methods may be used to control the surface energy of the capillary walls, such as coating with hydrophilic or hydrophobic materials, grafting, or corona treatments. For most passageways in the present invention the surface is generally hydrophilic since the liquid tends to wet the surface and the surface tension forces causes the liquid to flow in the passageway. For example, the surface energy of capillary passageways can be adjusted by known methods so that the contact angle of water is between 10° to 60° when the passageway is to contact whole blood or a contact angle of 25° to 80° when the passageway is to contact urine.

Movement of liquids through the capillaries typically is prevented by capillary stops, which, as the name suggests, prevent liquids from flowing through the capillary. If the capillary passageway is hydrophilic and promotes liquid flow, then a hydrophobic capillary stop can be used, i.e. a smaller passageway having hydrophobic walls. The liquid is not able to pass through the hydrophobic stop because the combination of the small size and the non-wettable walls results in a surface tension force which opposes the entry of the liquid. Alternatively, if the capillary is hydrophobic, no stop is necessary between a sample well and the capillary. The liquid in the sample well is prevented from entering the capillary until sufficient force is applied, such as by centrifugal force, to cause the liquid to overcome the opposing surface tension force and to pass through the hydrophobic passageway. It is a feature of such microfluidic chips that centrifugal force is only needed to start the flow of liquid. Once the walls of the hydrophobic passageway are fully in contact with the liquid, the opposing force is reduced because presence of liquid lowers the energy barrier associated with the hydrophobic surface. Consequently, the liquid no longer requires centrifugal force in order to flow. While not required, it may be convenient in some instances to continue applying centrifugal force while liquid flows through the capillary passageways in order to facilitate rapid analysis.

When the capillary passageways are hydrophilic, a sample liquid (presumed to be aqueous) will naturally flow through the capillary without requiring additional force. If a capillary stop is needed, one alternative is to use a narrower hydrophobic section which can serve as a stop as described above. A hydrophilic stop can also be used, even through the capillary is hydrophilic. Such a stop is wider and deeper than the capillary forming a “capillary jump” and thus the liquid's surface tension creates a lower force promoting flow of liquid. If the change in dimensions between the capillary and the wider stop is sufficient, then the liquid will stop at the entrance to the capillary stop. It has been found that the liquid will eventually creep along the hydrophilic walls of the stop, but by proper design of the shape this movement can be delayed sufficiently so that stop is effective, even though the walls are hydrophilic.

When a hydrophobic stop is located in a hydrophilic capillary, a pressure difference must be applied to overcome the effect of the hydrophobic stop. In general, pressure difference needed is a function of the surface tension of the liquid, the cosine of its contact angle with the hydrophilic capillary and the change in dimensions of the capillary. That is, a liquid having a high surface tension will require less force to overcome a hydrophobic stop than a liquid having a lower surface tension. A liquid which wets the walls of the hydrophilic capillary, i.e. it has a low contact angle, will require more force to overcome the hydrophobic stop than a liquid which has a higher contact angle. The smaller the hydrophobic channel, the greater the force which must be applied.

In order to design chips in which centrifugal force is applied to overcome hydrophilic or hydrophobic stops empirical tests or computational flow simulation can be used to provide useful information enabling one to arrange the position of liquid-containing wells on chips and size the interconnecting capillary channels so that liquid sample can be moved as required by providing the needed force by adjusting the rotation speed.

Microfluidic devices can take many forms as needed for the analytical procedures which measure the analyte of interest. The microfluidic devices typically employ a system of capillary passageways connecting wells containing dry or liquid reagents or conditioning materials. Analytical procedures may include preparation of the metered sample by diluting the sample, prereacting the analyte to ready it for subsequent reactions, removing interfering components, mixing reagents, lysising cells, capturing bio molecules, carrying out enzymatic reactions, or incubating for binding events, staining, or deposition. Such preparatory steps may be carried out before or during metering of the sample, or after metering but before carrying out reactions which provide a measure of the analyte.

### Applying Samples to Reagent Wells

Some wells will contain liquids for conditioning of a sample for reactions to indicate the presence and quantity of an analyte. In other wells, a liquid sample will be contacted with a reagent or conditioning agent supported on substrate such as a pad made of filter paper. In such cases, the reagent or conditioning agent is substantially dry or otherwise immobilized. The response depends on the amount and uniformity of the sample which is present and the amount of the component which responds to the reagent or conditioning agent. But, the response of a reagent or conditioning agent also depends on its access to the sample. If it is assumed that the regent or conditioning agent is distributed uniformly over a support so that the concentration of the reagent is the same at any place in the well, then the response of the reagent or conditioning agent to a uniform sample will also be uniform. That is, the overall response which is measured will be the sum of the response in each region of the well. However, if the sample itself is not uniform or the sample is not distributed uniformly over the reagent, then the overall measured response will not be accurate. For example, if, because all the air is not expelled from a well by the sample, some portion of the reagent will not respond to the sample. Or, if the sample is distributed over all of the reagent, but not uniformly, some regions will respond more strongly than other regions. The result is unlikely to be an accurate measure of the sample's content. The present invention provides a means of overcoming such difficulties.

It has been discovered that as samples become smaller, the introduction of liquid samples to reagent-containing substrates becomes more difficult. When it is possible to cover the reagent-containing substrate quickly with a large amount of liquid relative to the amount of the reagent, then it may not be important to provide features which direct the sample uniformly throughout the pad. However, in many instances it has been found that entry of the sample is critical to obtaining accurate and reliable analytical results.

Consider the typical substrate on which one or more reagents has been deposited. Reaction with components in the sample produces a detectable response, such as a change in color, reflectance, transmission or absorbance at a wavelength in the UV, VIS, IR, or Near IR wavelengths; or changes in Raman, fluorescence, chemiluminescence or phosphorescence events; or electro-chemical signals transduction. If a large amount of the component in the sample is to be reacted, and particularly if the response is qualitative in nature, then distribution of the sample over the surface of the substrate is less important. But, if the amount of the component is small relative to the amount of reagent, then the response may not be uniform and therefore less accurately measured. The component may react at the edge of the substrate where it enters and be exhausted before it reaches other portions of the substrate. Or, it may be drawn into an absorbent substrate and produce a non-uniform response in the pad, again leading to less accurate measurements. Thus, it will be evident that in such situations, distribution of the sample should be made as uniform as possible in order to produce accurate and consistent results.

In other situations, the substrate is not expected to produce uniform response to the application of a liquid sample. Instead, the sample is to be absorbed at one end of an elongated reagent area and then migrate by capillary action through the reagent area, where it meets a sequence of reagents and produces differing responses. It will be evident that the liquid sample should not flow over the surface so that it bypasses the sequence of reagents. Nor, should the sample bypass all or part of the elongated reagent area by capillary action at the edges of the substrate. In such situations, the entry of the sample to the elongated reagent area must be carefully controlled.

The flow of liquids in microfluidic chips involves the use of capillary forces and in many situations some other means to cause flow of liquids, such as centrifugal force. A liquid sample is moved through capillary passageways from an inlet port to one or more chambers where the sample is measured, preconditioned by contact with wash liquids, buffers, and the like, and then reacted with reagents to produce the desired response. The capillary passages typically are smaller than the chambers which they connect. Thus, the sample will flow from a relatively narrow passage into a much wider chamber where, for example, the sample contacts an absorbent substrate containing a reagent. One can visualize a stream of liquid entering a relatively large chamber and contacting the edge or other region of the absorbent substrate, from which it spreads by capillary action. Clearly, the amount of the component in the sample to be reacted with the reagent, the speed of reaction, and the rate at which the sample spreads will affect the response. Ideally, the sample will be uniformly distributed throughout the absorbent substrate and uniformly reacted with the reagent. In many instances, this cannot be achieved without providing microstructures which direct the flow of the sample onto the absorbent substrate in a uniform manner. Alternatively, when the absorbent pad is a chromatographic strip, the sample must not be directed uniformly over the strip, but must be confined to contacting the leading edge of the strip. Achieving such results in an effective manner is the objective of the invention.

### Microstructures

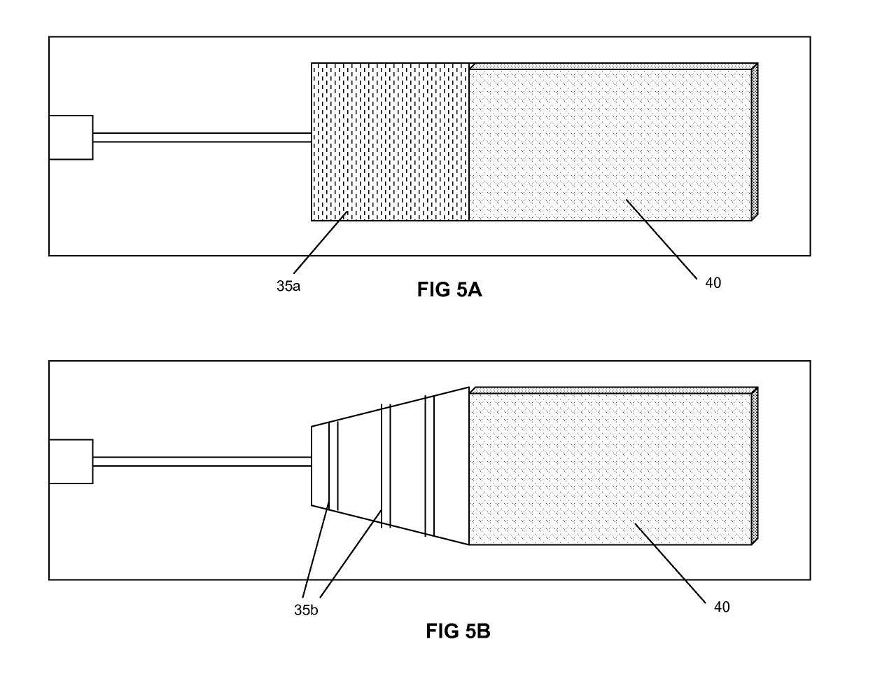

Referring to FIG. 5, one embodiment of a microfluidic chip containing microstructures and a separable reagent substrate according to the present invention is illustrated. The term “microstructures” as used herein relates to means for assuring that a microliter-sized liquid sample is most effectively contacted with a reagent or conditioning agent which is not liquid, but which has been immobilized on a substrate. Typically, the reagents or conditioning agents will be liquids which have been coated on a porous support and dried. Distributing a liquid sample as needed and at the same time purging air from the well can be done with various types of microstructures. By “microstructures” we mean structural features created in microfluidic chips which direct the flow of the liquid sample to the reagent in a predetermined manner, rather than randomly. In contrast to “microstructures”, the term “substrate” as used herein refers to a solid material, either absorbent or non-absorbent, on which a reagent or conditioning agent has been deposited. The reagent containing substrates are separate from microstructures and may or may not be in contact with the microstructures. Such substrates may include materials such as cellulose, nitrocellulose, plastics such as polyamides and polyesters, glass and the like and made in the form of paper, film, membrane, fiber, etc., either in solid or porous form.

Two preferred microstructures can be seen in FIGS. 4 and 5. An array of posts is disposed so that the liquid has no opportunity to pass through the inlet chamber in a straight line. The liquid is constantly forced to change direction as it passes through the array of posts. At the same time, the dimensions of the spaces between the posts are small enough to produce capillary forces inducing flow of the liquid. Air is purged from the reagent area as the sample liquid surges through the array of posts. Other types of microstructures which are useful include three dimensional post shapes with cross sectional shapes that can be circles, stars, triangles, squares, pentagons, octagons, hexagons, heptagons, ellipses, crosses or rectangles or combinations. FIG. 4 also shows grooves or weirs that are disposed perpendicularly to the direction of liquid flow to provide a uniform liquid front. Microstructures with two dimensional shapes such as ramps leading up or down to reagents on plateaus are also useful. Such ramps may include grooves at a right angle to the liquid flow to assist moving liquid or be curved.

The number and position of the microstructures depends on the capillary force desired for a particular reagent as well as the direction and location that the fluid flow is to occur. Typically a larger number of microstructures increases the capillary flow. As few as one microstructure can be used.

The microstructure may or may not contain additional geometric features to aid direct flow toward the reagent. These geometries can include rounded, convex, or concave edges, indentations, or grooves as well as partial capillaries. For example each of the posts can contain one or more wedge-shaped cutouts which facilitate the movement of the liquid onto the substrate containing the reagent. Such wedge-shaped cutouts are shown in U.S. Pat. No. 6,296,126.

### Applications

Microfluidic devices of the invention have many applications. Analyses may be carried out on samples of many biological fluids, including but not limited to blood, urine, water, saliva, spinal fluid, intestinal fluid, food, and blood plasma. Blood and urine are of particular interest. A sample of the fluid to be tested is deposited in the sample well and subsequently measured in one or more metering wells into the amount to be analyzed. The metered sample will be assayed for the analyte of interest, including for example a protein, a cell, a small organic molecule, or a metal. Examples of such proteins include albumin, HbA1c protease, protease inhibitor, CRP, esterase and BNP. Cells which may be analyzed include E. coli, pseudomonas, white blood cells, red blood cells, h. pylori, strep a, chlamydia, and mononucleosis. Metals which are to be detected include iron, manganese, sodium, potassium, lithium, calcium, and magnesium.

In many applications, color developed by the reaction of reagents with a sample his measured. It is also feasible to make electrical measurements of the sample, using electrodes positioned in the small wells in the chip. Examples of such analyses include electrochemical signal transducers based on amperometric, impedimetric, potentimetric detection methods. Examples include the detection of oxidative and reductive chemistries and the detection of binding events.

There are various reagent methods which could be used in chips of the invention. Reagents undergo changes whereby the intensity of the signal generated is proportional to the concentration of the analyte measured in the clinical specimen. These reagents contain indicator dyes, metals, enzymes, polymers, antibodies, electrochemically reactive ingredients and various other chemicals dried onto substrates. They can be introduced into the reagent wells in the chips of the invention to overcome the problems encountered in analyses using reagent strips.

Separation steps are possible in which an analyte is reacted with reagent in a first well and then the reacted reagent is directed to a second well for further reaction. In addition a reagent can be re-suspensed in a first well and moved to a second well for a reaction. An analyte or reagent can be trapped in a first or second well and a determination of free versus bound reagent be made.

The determination of a free versus bound reagent is particularly useful for multizone immunoassay and nucleic acid assays. There are various types of multizone immunoassays that could be adapted to this device. In the case of adaption of immunochromatography assays, reagents filters are placed into separate wells and do not have to be in physical contact as chromatographic forces are not in play. Immunoassays or DNA assay can be developed for detection of bacteria such as Gram negative species (e.g. E. Coli, Enterobacter, Pseudomonas, Klebsiella) and Gram positive species (e.g. Staphylococcus Aureus, Enterococc) Immunoassays can be developed for complete panels of proteins and peptides such as albumin, hemoglobin, myoglobulin, α-1-microglobulin, immunoglobulins, enzymes, glycoproteins, protease inhibitors and cytokines. See, for examples: Greenquist in U.S. Pat. No. 4,806,311, Multizone analytical Element Having Labeled Reagent Concentration Zone, Feb. 21, 1989, Liotta in U.S. Pat. No. 4,446,232, Enzyme Immunoassay with Two-Zoned Device Having Bound Antigens, May 1, 1984.

Potential applications where dried reagents are resolubilized include, filtration, sedimentation analysis, cell lysis, cell sorting (mass differences) and centrifugal separation. Enrichment (concentration) of sample analyte on a solid phase (e.g. microbeads) can be used to improve sensitivity. The enriched microbeads could be separated by continuous centrifugation. Multiplexing can be used (e.g. metering of a variety of reagent chambers in parallel and/or in sequence) allowing multiple channels, each producing a defined discrete result. Multiplexing can be done by a capillary array comprising a multiplicity of metering capillary loops, fluidly connected with the entry port, or an array of dosing channels and/or capillary stops connected to each of the metering capillary loops. Combination with secondary forces such as magnetic forces can be used in the chip design. Particle such as magnetic beads are used as a carrier for reagents or for capturing of sample constituents such as analytes or interfering substances. Separation of particles by physical properties such as density (analog to split fractionation).

The first example below illustrates the invention used in carrying out an assay for measuring the glycated hemoglobin (HbA1c) content of a patient's blood which can indicate the condition of diabetic patients. The method used has been the subject of a number of patents, most recently U.S. Pat. No. 6,043,043. Normally the concentration of glycated hemoglobin is in the range of 3 to 6 percent. But, in diabetic patients it may rise to a level about 3 to 4 times higher. The assay measures the average blood glucose concentration to which hemoglobin has been exposed over a period of about 100 days. Monoclonal antibodies specifically developed for the glycated N-terminal peptide residue in hemoglobin A1c are labeled with colored latex particles and brought into contact with a sample of blood to attach the labeled antibodies to the glycated hemoglobin. Before attaching the labeled antibodies, the blood sample is first denatured by contact with a denaturant/oxidant e.g. lithium thiocyanate as described in Lewis U.S. Pat. No. 5,258,311. Then, the denatured and labeled blood sample is contacted with an agglutinator reagent and the turbidity formed is proportional to the amount of the glycated hemoglobin present in the sample. The total amount of hemoglobin present is also measured in order to provide the percentage of the hemoglobin which is glycated.

### Example 1

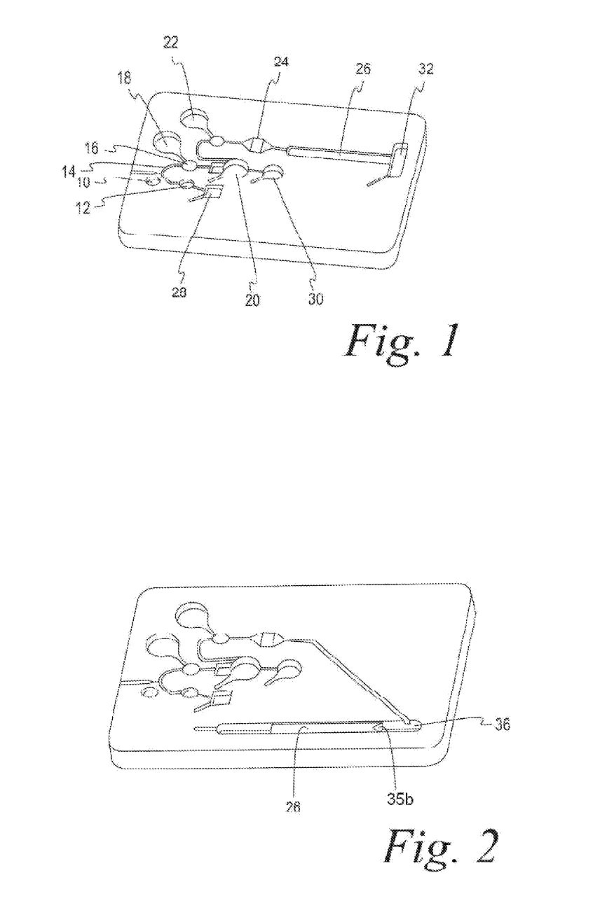

In this example, a test for HbA1c is carried out in a microfluidic chip of the type shown in FIG. 1. A sample of blood is introduced via sample port 10, from which it proceeds by capillary action to the pre-chamber 12 and then to metering capillary 14. The auxiliary metering well 16 is optional, only being provided where the sample size requires additional volume. The denaturant/oxidizing liquid is contained in well 18. Mixing chamber 20 provides space for the blood sample and the denaturant/oxidant well 22 contains a wash solution. Chamber 24 provides uniform contact of the preconditioned sample with labeled monoclonal antibodies disposed on a dry substrate. Contact of the labeled sample with the agglutinator, which is disposed on a substrate is carried out in chamber 26, producing a color which is measured to determine the amount of glycated hemoglobin in the sample. The remaining wells provide space for excess sample 28, excess denatured sample 30, and for a wicking material 32 used to draw the sample over the substrate in chamber 26.

A 2 μL sample was pipetted into sample port 10, from which it passed through a passageway located within the chip (not shown) and entered the pre-chamber 12, metering capillary 14, and auxiliary metering chamber 16. Any excess sample passes into overflow well 28, which contains a wetness detector. No centrifugal force was applied, although up to 400 rpm could have been used. The sample size (0.3 μL) was determined by the volume of the capillary 14 and the metering chamber 16. A capillary stop at the entrance of the capillary connecting well 16 and mixing well 20 prevented further movement of the blood sample until overcome by centrifugal force, in this example provided by spinning the chip at 700 rpm. The denaturant/oxidant solution lithium thiocyanate as described in Lewis U.S. Pat. No. 5,258,311 also was prevented from leaving well 18 by a capillary stop until 700 rpm was used to transfer 10 μL of the denaturant/oxidant solution along with the metered blood sample into mixing chamber 20. The volume of the mixing chamber 20 was about twice the size of the combined denaturant/oxidant solution and the blood sample. Then, the spinning speed was oscillated from about 100 to 1500 rpm to assure mixing of the liquids in chamber 20. After mixing, 2 μL of the mixture leaves mixing chamber 20 through a capillary and enters chamber 24 where microstructures assure uniform wetting of the substrate (a fibrous pad Whatman glass cellulose conjugate release paper) containing the latex labeled monoclonal antibodies for HbA1c. Incubation was completed within a few minutes, after which the labeled sample was released to agglutination chamber 26 by raising the rotation speed to 1300 rpm to overcome the capillary stop at the outlet of chamber 24. The labeled sample contacted the agglutinator (polyaminoaspartic acid HbA1c peptide) which was striped on a Whatman 5 μm pore size nitrocellulose reagent in concentrations of 0.1 to 5.0 mg/mL. The absorbent material (Whatman cellulose wicking paper) in well 32 facilitated uniform passage of the labeled sample over the strip. (Alternatively, centrifugal force could be used). Distribution of the labeled sample over the strip was provided by microstructures located at the inlet of chamber 26. Finally, the rotation speed was raised to 2500 rpm to overcome a capillary stop preventing the wash solution from leaving well 22. The buffer solution (phosphate buffered saline) passes through chamber 24 and over the strip in chamber 26 to improve the accuracy of the reading of the bands on the strip. The color developed was measured by reading the reflectance with a digital camera, scanner or other reflectometer such a Bayer CLINITEK instrument.

Results for such measurements are illustrated in the following table.

TABLE

| | |
|---|---|
| HbA1c | Peak Height (% R) |

| (μm) | Mean | SD |
|---|---|---|
| 346.12 | 16.6 | 0.4 |
| 391.75 | 13.0 | 0.5 |
| 437.34 | 11.1 | 1.0 |
| 482.96 | 8.6 | 0.3 |
| 528.57 | 6.3 | 0.6 |
| 574.18 | 3.9 | 0.5 |

### Example 2

The test described in Example 1 was repeated, using the modified microfluidic chip shown in FIG. 2. In FIG. 2, the agglutinator chamber 26 was positioned so that the labeled sample flowed “uphill”, i.e. toward the center of rotation, assisted by the wicking action of absorbent material placed at the uphill end of the strip. Equivalent results were obtained. In this case, the microstructure that directs the flow is a ramp 35b leading upward to a plateau onto which the nitrocellulose reagent is placed. In an alternative embodiment, the strip would extend into the pre-chamber 36 which contains the sample liquid.

### Example 3

The test of Example 1 is repeated with a microfluidic chip in which the labeled sample entered at the center of the agglutination strip 26 so that the labeled sample wicks in two directions.

### Example 4

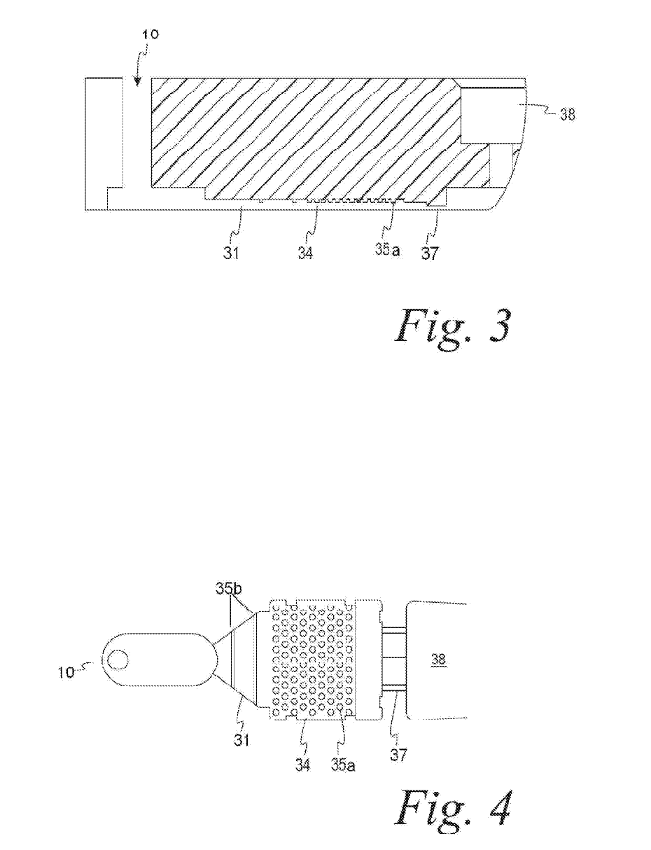

The invention is further illustrated in FIGS. 3 and 4, which show a microfluidic device, one of many disposed on a sample disc for measurement of glucose in blood. In the sectional view of FIG. 3, a sample of blood is deposited in entry port 10 from which it flows by capillary action down through an inlet passageway 31 containing ridges and grooves 35b disposed perpendicularly to the flow of the sample in order to create a uniform liquid front and allowing the same capillary force to be applied across the reagents edge. The passageway 31 fans out until it reaches chamber 34, which contains microstructures to facilitate contact with the chromogenic glucose reagent disposed on a porous substrate (as described in Bell U.S. Pat. No. 5,360,595). FIG. 4 illustrates the array of microstructure posts 35a used. As the sample enters the reagent chamber 34, air is purged through several capillary passages 37, exiting through outlet 38.

The microfluidic device of FIG. 3 was used to measure the glucose content of blood. Whole blood pretreated with heparin was incubated at 250° C. to degrade glucose naturally occurring in the blood sample. The blood was spiked with 0, 50, 100, 200, 400, and 600 mg/μL of glucose as assayed on the glucose reference assay instrument (YSI Inc.). A glucose reagent (as described in Bell U.S. Pat. No. 5,360,595) was coated on a nylon membrane (Biodyn from Pall Corp) disposed on a plastic substrate. A sample of the reagent on its substrate (not shown) was placed in chamber 34 in contact with microstructures 35a and the bottom of the device covered with Pressure sensitivity adhesive lid Sealplate from Excel.

Samples of blood containing one of the concentrations of glucose were introduced into inlet port 30 using a 2 μL capillary with plunger (AquaCap from Drummond Inc). Since the inlet port is sealed when the sample is dispensed, a positive pressure is established which forces the sample into the inlet passageway 31 and then into the reagent area 34. The sample reacted with the reagent to provide a color, which is then read on a spectrometer at 680 nm, as corrected against a black and white standard.

Additionally two plastic substrates, PES and PET, were used with the series of blood samples. Where PET coated with reagent were used, a 500 nm to 950 nm transmittance meter was used to read the reaction with the sample. Where PES coated with reagent was used a bottom read reflectance meter (YSI instrument) was used to read the reaction with the sample.

Comparable results were obtained, as can be seen in the following table.

TABLE 2

| Expected | Observed |
|---|---|
| Glucose | Glucose (n = 6) |
| 0 | 0.3 |
| 50 | 48.5 |
| 100 | 103.1 |
| 200 | 197.3 |
| 400 | 409.1 |
| 600 | 586.7 |

### Comparative Example

The experiment of Example 4 was repeated with the reagent area 34 having no microstructures to provide uniform contact with the reagent. It was found that the reagent well could not be filled completely and portions were unfilled because air was not expelled.

### Example 5

The tests of Example 4 were repeated without using positive pressure at the entry port 10 to push the sample into the reagent chamber. Instead, a vacuum was applied at the exit port 38. Equivalent results were obtained.

What is claimed is:

1. A microfluidic device for assaying a liquid biological sample of 10 μL or less,
said device including a space containing microstructures in which a reagent or conditioning agent is immobilized on a substrate and positioned separately from said microstructures
said reagent or conditioning agent deposited on said substrate as a liquid and dried.

2. A microfluidic device of claim 1 wherein said substrate is an absorbent or non-absorbent solid.

3. A microfluidic device of claim 2 wherein said substrate is in the form of paper, film, membrane, or fiber.

4. A microfluidic device of claim 3 said substrate comprises cellulose, nitrocellulose, polyamides, polyesters or glass.

5. A microfluidic device of claim 1 wherein said microstructures are an array of posts positioned adjacent said substrate and aligned at right angle to the flow of said sample.

6. A microfluidic device of claim 1 wherein said microstructures include at least one of the group consisting of ramps, grooves, and weirs to direct flow to said substrate.

7. A microfluidic device of claim 5 wherein said array of posts includes at least two columns or posts staggered to prevent said sample from flowing in a straight line through said space.

8. A microfluidic device of claim 5 wherein said posts have at least one wedge-shaped cutout aligned vertically to said substrate for facilitating movement of the sample onto said substrate.

9. A microfluidic device of claim 5 wherein said substrate is positioned above or below array of posts.

10. A microfluidic device of claim 6 wherein said microstructure is a ramp for directing flow of the sample upward or downward to said substrate disposed on a plateau of said space.

11. A microfluidic device of claim 6 wherein said microstructure is a groove or weir disposed at a right angle to the flow of said sample.

12. A microfluidic device of claim 11 where said groove or weir have at least one wedge-shaped cutout for facilitating distribution of the sample.

13. A microfluidic device for assaying a liquid biological sample of 10 μL or less comprising:
(a) an inlet port for receiving said sample;
(b) a capillary passageway in fluid communication with said inlet port, said passageway having dimensions that induce a predetermined capillary force for moving said sample through said passageway;
(c) a well defined by top and bottom surfaces enclosing a side wall, said well having an entry in said sidewall for introducing said sample into said well from said capillary passageway and an air vent positioned on said sidewall, said well containing microstructure for directing flow of said sample;
(d) a reagent or conditioning agent immobilized on a substrate positioned separately said microstructures.

14. A microfluidic device of claim 13 wherein said substrate us an absorbent or non-absorbent solid in the form of paper, films, membrane or fiber.

15. A microfluidic device of claim 14 wherein said substrate comprises cellulose, nitrocellulose, polyamides, polyesters or glass.

16. A microfluidic device of claim 13 wherein said microstructures of claim 13 wherein said microstructures are an array of posts positioned adjacent said substrate and aligned at right angle to the flow of said sample.

17. A microfluidic device of claim 16 wherein said posts have at least one wedge-shaped cutout aligned vertically to said substrate for facilitating movement of the sample onto said substrate.

18. A microfluidic device of claim 13 wherein said microstructures include at least one of the group consisting ramps, grooves, and weirs to direct flow to said substrate.

19. A microfluidic device of claim 18 wherein said microstructure is a ramp for directing flow of the sample upward or downward to said substrate disposed on a plateau of said well.

20. A microfluidic device of claim 18 wherein said microstructure is a groove or weir disposed at a right angle to the flow of said sample.
